(19) United States
(12) Patent Application Publication
(10) Pub. No.: US 2023/0174858 A1
(43) Pub. Date: Jun. 8, 2023

(54) UPCONVERTING NANOPARTICLES

(71) Applicants: University of Pittsburgh-Of the Commonwealth System of Higher Education, Pittsburgh, PA (US); REGENTS OF THE UNIVERSITY OF MINNESOTA, Minneapolis, MN (US)

(72) Inventors: Renee Frontiera, Minneapolis, MN (US); Jill Millstone, Pittsburgh, PA (US); Chris Warkentin, Minneapolis, MN (US); Xing Yee Gan, Pittsburgh, PA (US); Emily Keller, Minneapolis, MN (US)

(21) Appl. No.: 17/923,824

(22) PCT Filed: May 7, 2021
(86) PCT No.: PCT/US2021/031399

§ 371 Date: Nov. 7, 2022

Related U.S. Application Data
(60) Provisional application No. 63/021,413, filed on May 7, 2020.

(51) Int. Cl. C09K 11/88; C09K 11/02; A61K 41/00; A61K 9/51; H01G 9/20; H10K 30/35; H10K 30/87; H01L 31/055

(52) U.S. Cl. CPC C09K 11/881; C09K 11/02; A61K 41/0057; A61K 9/5115; H01G 9/2009; H01G 9/209; H10K 30/35; H10K 30/87; H01L 31/055; B82Y 20/00

(57) ABSTRACT

A device includes chalcogenide nanoparticles and a light-sensitive material configured to absorb upconverted light generated by the chalcogenide nanoparticles. A method includes receiving, at chalcogenide nanoparticles, input light having a first wavelength; and upconverting the input light using the chalcogenide nanoparticles, to generate output light having a second wavelength, in which the second wavelength is less than the first wavelength. A device includes a transparent material, the transparent material being transparent to at least one of infrared light and visible light, and chalcogenide nanoparticles embedded in the transparent material.

### CROSS-REFERENCE TO RELATED APPLICATIONS

This application claims the benefit of U.S. Provisional Application Ser. No. 63/021,413, filed May 7, 2020. The disclosure of the prior application is considered part of (and is incorporated by reference in) the disclosure of this application.

### STATEMENT AS TO FEDERALLY SPONSORED RESEARCH

This invention was made with government support under FA9550-15-1-0022 awarded by the Air Force Office of Scientific Research and under HDTRA1-16-1-0044, awarded by the Defense Threat Reduction Agency. The government has certain rights in the invention.

### BACKGROUND

Photon upconversion is the transformation of lower-energy light into higher-energy light. Photon upconversion using suitable upconverting materials has the potential to be applicable in a variety of fields.

### SUMMARY

The present disclosure describes particles for use in photon upconversion including, in particular, chalcogenide nanoparticles. In general, in some aspects, the subject matter of the present disclosure is directed to devices that include chalcogenide nanoparticles and a light-sensitive material configured to absorb upconverted light generated by the chalcogenide nanoparticles.

In some implementations, the chalcogenide nanoparticles are copper selenide nanoparticles. In some implementations, the copper selenide nanoparticles are hole-doped Cu₂₋ₓSe nanoparticles.

In some implementations, the chalcogenide nanoparticles are embedded in a solid layer transparent to at least one of infrared light and visible light. In some implementations, the solid layer includes a glass or a polymer. In some implementations, an absorber layer includes the light-sensitive material, and the solid layer is disposed on the absorber layer. In some implementations, the solid layer is disposed apart from the light-sensitive material. In some implementations, the device includes an anti-reflection coating on the solid layer.

In some implementations, the light-sensitive material is a light absorber in a solar cell or a photosensor. In some implementations, the chalcogenide nanoparticles each have a diameter between about 10 nanometers and about 30 nanometers. In some implementations, the chalcogenide nanoparticles upconvert infrared light into visible light.

In some implementations, the chalcogenide nanoparticles include ligands or small molecules bound to the chalcogenide nanoparticles. In some implementations, the chalcogenide nanoparticles are embedded in the light-sensitive material. In some implementations, the light-sensitive material is a perovskite light absorber. In some implementations, the light-sensitive material is an organic light absorber. In some implementations, the device is part of a solar cell or a photosensor. In some implementations, the chalcogenide nanoparticles are suspended in a liquid solvent.

In some implementations, the chalcogenide nanoparticles are bound to a coating suitable for placement into a patient undergoing medical treatment. In some implementations, the coating is configured to bind to a cell inside the patient. In some implementations, the coating is bound to a drug. In some implementations, the device includes a light source configured to illuminate the chalcogenide nanoparticles with infrared light while the chalcogenide nanoparticles are inside the patient. In some implementations, the light source is a continuous-wave laser. In some implementations, the chalcogenide nanoparticles are configured to upconvert infrared light generated by the light-sensitive material into visible light absorbed by the light-sensitive material.

In general, in some aspects, the subject matter of the present disclosure is directed to methods that include receiving, at chalcogenide nanoparticles, input light having a first wavelength; and upconverting the input light using the chalcogenide nanoparticles, to generate output light having a second wavelength, in which the second wavelength is less than the first wavelength.

In some implementations, the input light is infrared light, and the output light is visible light. In some implementations, the method includes absorbing the output light at a light absorber of a photovoltaic device or a photodetector device. In some implementations, the method includes illuminating the chalcogenide nanoparticles with the input light, and receiving the output light at an imaging device.

In some implementations, the chalcogenide nanoparticles are inside a patient undergoing medical treatment. In some implementations, the chalcogenide nanoparticles are bound to a coating configured to bind to a cell inside the patient.

In some implementations, the chalcogenide nanoparticles are copper selenide nanoparticles. In some implementations, the copper selenide nanoparticles are hole-doped Cu₂₋ₓSe nanoparticles. In some implementations, the chalcogenide nanoparticles are embedded in a solid layer transparent to at least one of infrared light and visible light. In some implementations, the chalcogenide nanoparticles each have a diameter between about 10 nanometers and about 30 nanometers. In some implementations, the chalcogenide nanoparticles include ligands or small molecules bound to the chalcogenide nanoparticles.

In some aspects, the subject matter of the present application is directed to devices that include a transparent material, the transparent material being transparent to at least one of infrared light and visible light, and chalcogenide nanoparticles embedded in the transparent material. In some implementations, the transparent material includes a glass. In some implementations, the glass includes SiO₂ derived from a sol-gel process. In some implementations, the transparent material includes a polymer. In some implementations, the polymer includes at least one of polyvinylpyrrolidone or polystyrene. In some implementations, the device includes a frame containing the transparent material, in which the frame is configured to be attached to a solar module. In some implementations, an internal quantum yield of the chalcogenide nanoparticles embedded in the transparent material is at least about 2% for 1064 nm input light. In some implementations, an onset power density of the chalcogenide nanoparticles embedded in the transparent material is less than about 1000 W/cm² for 1064 nm input light.

Particular embodiments of the subject matter described in this specification can be implemented to realize one or more of the following advantages. For example, in some implementations, the photon upconverters of the present disclosure upconvert input light of lower intensities compared to other upconverters. In some implementations, the photon upconverters upconvert light with a greater efficiency compared to other photon upconverters. In some implementations, the photon upconverters upconvert light generated by low-threshold and/or continuous-wave light sources. In some implementations, the photon upconverters upconvert light with a lower upconversion threshold compared to other upconverters. In some implementations, the light-sensitive material may be configured particularly to absorb light generated by the photon upconverters. In some implementations, the photon upconverters may be configured particularly to upconvert light to be absorbed by the light-sensitive material. In some implementations, the photon upconverters may be less toxic compared to other photon upconverters. In some implementations, the photon upconverters may be more air- and/or moisture-stable compared to other upconverters. In some implementations, the photon upconverters may upconvert light over a greater wavelength range compared to other upconverters. In some implementations, the photon upconverters may be configurable (e.g., by a tunable size) to upconvert a particular range of wavelengths of light. In some implementations, the photon upconverters may be configurable to generate upconverted light having wavelengths within a particular range of wavelengths of light.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a schematic showing an example of upconverting chalcogenide nanoparticles and a light-sensitive material.

FIG. 2 is a chart showing an example of an upconverted light spectrum.

FIG. 3 is a schematic showing an example of an upconverting chalcogenide nanoparticle bound to a compound.

FIG. 4 is a schematic showing an example of upconverting chalcogenide nanoparticles embedded in a solid layer.

FIG. 5 is a schematic showing an example of upconverting chalcogenide nanoparticles integrated into a frame.

FIG. 6 is a schematic showing an example of upconverting chalcogenide nanoparticles embedded in a light-absorbing layer.

FIG. 7 is a schematic showing an example of upconverting chalcogenide nanoparticles inside a patient.

FIGS. 8A-8B are charts showing upconversion data of upconverting chalcogenide nanoparticles embedded in a solid layer.

FIG. 9 is a flowchart showing an example method according to some implementations of this disclosure.

### DETAILED DESCRIPTION

The present disclosure relates to photon upconverting materials. In particular, in certain implementations, this disclosure describes chalcogenide nanoparticles that upconvert light for absorption by a light-sensitive material.

Photon upconversion (or simply “upconversion”) is a process in which lower-energy photons are converted into higher-energy photons. In some cases, two lower-energy photons are converted into a higher-energy photon having an energy that is the sum of energies of the lower-energy photons. Upconversion has applications in a variety of fields, including solar energy harvesting, photodynamic therapy, thermal management strategies, and deep-tissue imaging.

However, existing upconverting materials and devices often require high input light intensities in order to generate appreciable output light, in some cases due to the non-linearity of multi-photon processes. In some cases, this high minimum input light intensity is comparable to the intensity of light emitted by pulsed, rather than continuous-wave, lasers. Many applications are incompatible with this intensity requirement: for example, unconcentrated sunlight has relatively low intensity, and, in medical applications, tissue may be damaged by high-intensity light.

In addition, existing upconverting materials may be toxic, making them undesirable for widespread use.

This disclosure describes upconversion using, e.g., chalcogenide nanoparticles capable of upconverting low-intensity light, such as unconcentrated solar output or the output of a low threshold, continuous wave laser.

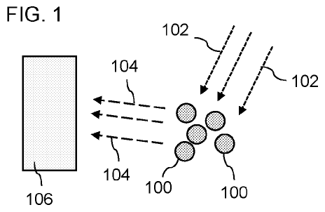

As shown in FIG. 1, chalcogenide nanoparticles 100 (an example of a photon upconverter) are illuminated by input light 102 having a first wavelength. The chalcogenide nanoparticles perform upconversion, resulting in the generation and emission of output light 104 having a second wavelength lower than the first wavelength. A light-sensitive material 106 absorbs the output light 104.

The difference in wavelength between the input light 102 and the output light 104 is caused at least by plasmonic effects of the chalcogenide nanoparticles 100. Charge carriers within each nanoparticle oscillate collectively under the stimulation of the input light 102, leading to scattering through plasmon resonance. The strength of the interaction between the input light 102 and the collective oscillation of the charge carriers causes efficient conversion of pairs of lower-energy photons in the input light 102 into single higher-energy photons in the output light 104.

In some implementations, the light-sensitive material 106 is more sensitive to the output light 104 than to the input light 102. For example, the light-sensitive material 106 may absorb light having the second wavelength more strongly (for example, with a higher absorption coefficient) than the light-sensitive material absorbs light having the first wavelength.

Many solar cell light-absorber materials (for example, silicon) absorb light strongly below a wavelength corresponding to the material's bandgap, while absorbing higher-wavelength light more weakly. This leads to a loss in photovoltaic efficiency as higher-wavelength (lower-energy) light present in the solar spectrum is not absorbed. If the higher-wavelength light is upconverted into lower-wavelength light, as shown in FIG. 1, then the light-sensitive material 106 (for example, the silicon absorber in a silicon solar cell) is more likely to absorb the light, and overall photovoltaic conversion efficiency may be improved.

The light-sensitive material 106 may be configured and/or selected to absorb the output light 104. For example, the light-sensitive material 106 may be positioned near the chalcogenide nanoparticles 100 in order to absorb the output light 104. The light-sensitive material 106 may be positioned to have a surface 108 facing the chalcogenide nanoparticles 100 in order to absorb the output light 104. The light-sensitive material 106 may have a bandgap such that the light-sensitive material absorbs the output light 104 more strongly than the light-sensitive materials absorbs the input light 102. The light-sensitive material 106 may exhibit an absorption peak such that the light-sensitive material absorbs the output light 104 more strongly than the light-sensitive material absorbs the input light 102.

Besides chalcogenide nanoparticles, which are used as an example throughout this disclosure, other photon upconverters may also be included in the devices, methods, and systems described in this disclosure. For example, the photon upconverters may include chalcogenide nanostructures besides nanoparticles, e.g., chalcogenide nanostructures on the surface of a substrate or embedded in layers formed on a substrate. Upconverting chalcogenide nanostructures may have the dimensions, chemistry, features, and advantages disclosed herein for the example of upconverting chalcogenide nanoparticles, including the disclosed relationships between the chalcogenide nanoparticles and the light-sensitive material. Upconverting chalcogenide nanostructures may be included in devices as described below for upconverting chalcogenide nanoparticles, e.g., included in photovoltaic devices and sensing devices.

“Nanoparticles” and “nanostructures” as described in this disclosure include, in some implementations, particles and structures having dimensions beyond the nanometer range. For example, in some implementations, photon upconverter nanoparticles and nanostructures may have dimensions of microns, tens of microns, hundreds of microns, or larger than hundreds of microns. In some implementations, devices may include arrays or assemblies of nanoparticles.

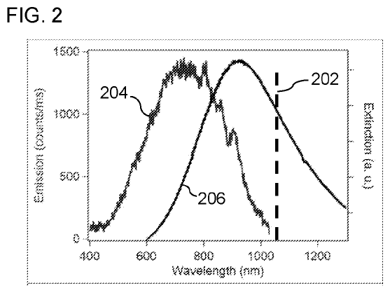

FIG. 2 shows an example of upconversion by copper selenide chalcogenide nanoparticles. An input excitation 202 at 1064 nm (from a continuous-wave laser) is upconverted into a broadband output signal 204 by the chalcogenide nanoparticles. The output signal 204 has spectral characteristics (for example, a full-width at half-maximum) approximately matching spectral characteristics of the plasmon resonance 206.

In some implementations, the chalcogenide nanoparticles upconvert light over a broad wavelength range, for example, over a range of more than 1000 nanometers (nm), over a range of more than 500 nm, over a range of more than 300 nm, over a range of more than 200 nm, or over another range. The range may be less than 2000 nm, less than 1500 nm, less than 1000 nm, less than 750 nm, less than 500 nm, or a another range. In some implementations, the chalcogenide nanoparticles upconvert light substantially uniformly over a wavelength range, such that relative intensities of light within an input wavelength range are preserved in relative intensities of light within an output wavelength range.

In some implementations, as shown in FIG. 2, the chalcogenide nanoparticles upconvert infrared light into visible light. In some implementations, the chalcogenide nanoparticles upconvert infrared light and/or visible light into ultraviolet light. In some implementations, the chalcogenide nanoparticles upconvert light within a spectrum of light, e.g., upconvert lower-energy visible light into higher-energy visible light, or upconvert shortwave-infrared light into higher-energy infrared light.

In some implementations, the photon upconverters are bound to one or more compounds. “Compounds,” as used in this disclosure, refers at least to small molecules and assemblies of small molecules, polymers, particles (e.g., nanoparticles and/or microparticles), and structures comprising a plurality of particles, chemical compounds and/or components. The compounds may be, for example, ligands and/or small molecules.

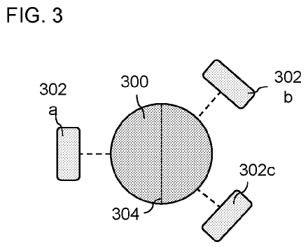

For example, as shown in FIG. 3, a chalcogenide nanoparticle 300 is bound to compounds 302a, 302b, 302c. In some implementations, the nanoparticle 300 is bound to an organic compound, for example, oleylamine or a polymer.

In some implementations, the nanoparticle 300 is bound to an inorganic compound. Inorganic compounds bound to the nanoparticle 300 may include one or more of transition metal oxides, metals and semi-metals, two-dimensional materials, and oxides (e.g., silicon oxide). In some implementations, the compounds form an oxide support or a shell.

The nanoparticle 300 may be terminated with (e.g., bound to) a compound that allows binding to a second compound. For example, if the nanoparticle 300 is embedded in a solid, the nanoparticle may be bound to an intermediate compound that binds to the solid (e.g., binds to a polymer that makes up the solid). In some implementations, the nanoparticle 300 may be directly bound (e.g., through chemical binding) to the solid in which the nanoparticle 300 is embedded. The nanoparticle 300 may be terminated with a compound that allows the nanoparticle 300 to be suspended in solution.

The compounds 302a, 302b, 302c may form a coating (e.g., a shell) around the nanoparticle 300. In some implementations, the coating is non-toxic (e.g., for ingestion or insertion into a patient). In some implementations, the coating serves to protect the nanoparticle 300, e.g., from chemical reactions with external chemicals.

In some implementations, the chalcogenide nanoparticles are selenium-based nanoparticles, for example, copper selenide nanoparticles. In some implementations, the chalcogenide nanoparticles are sulfur-based or tellurium-based, for example, iron sulfide nanoparticles or lead telluride nanoparticles. In some implementations, the chalcogenide nanoparticles include multiple chalcogen elements, for example, both sulfur and selenide.

In the case of copper selenide nanoparticles, these nanoparticles may self-dope (spontaneously or, for example, upon exposure to oxygen). In the self-doping process, some Cu⁺ ions in the nanoparticles are oxidized to Cu²⁺, which results in the formation of holes in the valence band. Self-doped copper selenide can be referred to as Cu₂₋ₓSe, with “2-x” (1≤x≤2) indicating the varied oxidation states of the copper in the nanoparticles. “Copper selenide” as used in this disclosure refers at least to compounds of copper and selenium, including Cu₂₋ₓSe, without indicating a specific stoichiometry or oxidation state of those elements in the compounds.

Compared to other upconverters, the chalcogenide nanoparticles disclosed herein may have enhanced upconversion efficiencies. For example, an upconversion threshold (e.g., a threshold power density of input light greater than which upconversion efficiency increases non-linearly) of the chalcogenide nanoparticles may be lower than for other upconverters. In some implementations, an upconversion threshold may be less than about 9 kW/cm² for a sample with about 10⁵ chalcogenide nanoparticles in the interaction volume.

An upconversion efficiency (over at least a portion of the electromagnetic spectrum) of the chalcogenide nanoparticles may be higher than for other upconverters. A higher upconversion efficiency may allow the chalcogenide nanoparticles to be used in applications in which input light is not expected to be of high intensity (for example, photovoltaic applications or medical applications).

In addition, the chalcogenide nanoparticles may be non-toxic and stable compared to other upconverters. For example, the chalcogenide nanoparticles may be air- and/or moisture-stable, such that they do not degrade over time when embedded into devices, or degrade less over time than other upconverters do. The non-toxicity of the chalcogenide nanoparticles may allow them to be used for medical applications, as described below.

Compared to other upconverters, the chalcogenide nanoparticles may be less expensive or otherwise easier to produce. For example, the chalcogenide nanoparticles may include earth-abundant materials, while other upconverters may include rarer or more expensive materials.

The chalcogenide nanoparticles may upconvert with spectral characteristics that other upconverters may not have. For example, the chalcogenide nanoparticles may upconvert from a particular first wavelength range (e.g., infrared) to a particular second wavelength range (e.g., visible), while other upconverters may upconvert to or from other wavelength ranges. The chalcogenide nanoparticles may not interact with light, or interact less with light, in a particular wavelength range (e.g., visible), while other upconverters may interact more strongly with light in that particular wavelength range. The chalcogenide nanoparticles may upconvert over a broader wavelength band than do other upconverters.

The chalcogenide nanoparticles may have the upconversion properties and advantages described herein at least because of the chalcogenide nanoparticles' broadband and tunable plasmon resonance extending across the near infrared region. In some implementations, the plasmon resonance is tunable by varying the size (e.g., diameter) of the nanoparticles. In some implementations, due to the fast nature of the coherent plasmon decay exhibited by stimulated chalcogenide nanoparticles, the multiphoton upconversion process arises from interactions with numerous hot carriers generated in the chalcogenide nanoparticles, enabling efficient upconversion with weak excitation.

Oxygen may be present in the nanoparticles, for example, near the outer interfaces of the nanoparticles.

Details on the synthesis and structure of copper selenide nanoparticles can be found in Gan et al., “Plasmon-Enhanced Chemical Conversion Using Copper Selenide Nanoparticles,” Nano Letters 2019 19 (4), 2384-2388, and the Supporting Information thereof, both of which are incorporated herein by reference in their entirety.

The chalcogenide nanoparticle 300 has a diameter 304. In some implementations, the chalcogenide nanoparticles have diameters between about 5 and about 50 nm. In some implementations, the chalcogenide nanoparticles have diameters between about 10 and about 30 nm. In some implementations, the chalcogenide nanoparticles have diameters between about 10 and about 20 nm. In some implementations, the chalcogenide nanoparticles have diameters less than about 200 nm. In some implementations, the chalcogenide nanoparticles have diameters less than about 1000 nm. In some implementations, the chalcogenide nanoparticles have diameters less than about 5000 nm.

FIG. 3 shows the nanoparticle 300 as substantially circular/spherical. However, the photon upconverters may have a variety of shapes, including, for example, ovoids, pillars, and ridges. When the photon upconverters are not spherical, the aforementioned dimensions of the diameter may equally refer to another dimension of the photon upconverters.

In some implementations, the chalcogenide nanoparticles have diameters tuned to give particular upconversion properties. For example, a wavelength-dependent efficiency and/or degree of upconversion may be determined at least in part by the diameter. The diameter may be tuned to set a wavelength difference between input light and output light, or between an output light wavelength band and an input light wavelength. The diameter may be tuned to set spectral characteristics of the output light. For example, the diameter may be tuned to set a wavelength maximum for upconverted light.

The diameter may be tuned such that upconverted light generated by the chalcogenide nanoparticles is absorbed more strongly by the light-sensitive material than the input light is absorbed by the light-sensitive material. The diameter may be tuned such that the nanoparticles do not interact strongly with a particular wavelength range of input light.

In some implementations, chalcogenide nanoparticles having more than one diameter are used. For example, chalcogenide nanoparticles having different diameters may upconvert different portions of the electromagnetic spectrum of input light, either exclusively or with varying efficiencies. In some implementations, the chalcogenide nanoparticles having varied diameters may be interspersed evenly throughout a solid layer of a device. In some implementations, the chalcogenide nanoparticles may be distributed in one or more stacked layers such that input light of different wavelengths is upconverted (or is more likely to be upconverted) in a specific layer of the stacked layers, corresponding to the size of the chalcogenide nanoparticles in that layer. The inclusion of chalcogenide nanoparticles of different diameters may allow the efficient upconversion of a broader range of the electromagnetic spectrum than would be caused by only a single size of chalcogenide nanoparticle.

As another example, chalcogenide nanoparticles having different diameters may be used to upconvert input light into output light having two or more distinct peaks, spectral shapes, and/or wavelength difference from the input light. Each peak, spectral shape, and/or wavelength difference from the input light may correspond to a particular diameter of chalcogenide nanoparticle.

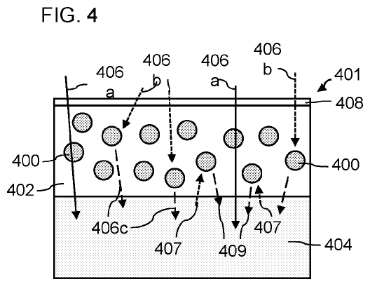

FIG. 4 shows an example device 401 including chalcogenide nanoparticles 400. The nanoparticles 400 are embedded in (e.g., layered in and/or dispersed in) a solid layer 402. The solid layer 402 is disposed on an absorber layer 404 that includes light-sensitive material. In some implementations, the absorber layer 404 is entirely the light-sensitive material.

Input light of a variety of wavelengths is incident on the device 401. Some input light 406a (for example, visible light) is transmitted through the solid layer 402 without interacting substantially with the solid layer 402 and the nanoparticles 400. Other light 406b (for example, infrared light) is upconverted by the nanoparticles 400 into output light 406c, which is then transmitted through the solid layer 402 to the absorber layer 404. Transmitted light 406a and 406c is then absorbed in the absorber layer 404.

In some implementations, the absorber layer 404 is a light absorber in a solar cell. Light absorption by the light-sensitive material within the absorber layer 404 causes energy generation. However, most light-sensitive materials (for example, silicon and other inorganic semiconductors, organic semiconductors, and hybrid organic-inorganic perovskites) do not absorb all portions of the solar spectrum equally strongly. In the absence of the solid layer 402 including the chalcogenide nanoparticles 400, some input light (e.g., infrared input light 406b) may pass through the solar cell without being absorbed by the absorber layer 404, leading to decreased photovoltaic conversion efficiencies.

However, upconversion by the nanoparticles 400 can convert input infrared light 406b into output visible light 406c, which is more readily absorbed by the light-sensitive material in the absorber layer 404. Therefore, overall photovoltaic conversion efficiency may be increased.

In some implementations, the solid layer 402 is substantially transparent to at least one of infrared light and visible light. This may allow the absorber layer 404 to absorb additional light, rather than having that light be absorbed by the solid layer 402 itself. Note that the solid layer 402 need not be transparent across the entire infrared range and/or visible range. For example, in some implementations the solid layer 402 is transparent in the near-infrared range, such as in a portion of the near-infrared range. “Transparent” may include layers that transmit 98% of light to which the layers are transparent, 95% of light to which the layers are transparent, 90% of light to which the layers are transparent, 80% of light to which the layers are transparent, 70% of light to which the layers are transparent, or another percentage of light to which the layers are transparent.

In some implementations, the absorber layer 404 is a light-receiving layer in a photosensor. For example, the device 401 may be an infrared camera using visible-light photosensors. Input infrared light is upconverted into visible light capable of being efficiently collected by the absorber layer 404 of the photosensor.

The solid layer 402 may be, for example, a coating or a film deposited on the absorber layer 404. In some implementations, the nanoparticles 400 are dispersed in the solid layer 402 prior to deposition (e.g., prior to spin-coating a sol-gel solution from which the solid layer 402 will be derived onto the absorber layer 404). In some implementations, the solid layer 402 includes one or more of an oxide (for example, SiO₂/glass, e.g., a sol-gel derived SiO₂ layer), a glass, a nitride such as silicon nitride, or a polymer. For example, the nanoparticles 400 may be embedded in one or more polymers such as polyvinylpyrrolidone and/or polystyrene.

In some implementations, an anti-reflection coating 408 is formed as part of, or on top of, the solid layer 402. For example, the solid layer 402 may be textured with anti-reflection surface elements.

In some implementations, the device 401 includes a reflection layer (e.g., between the solid layer 402 and the absorber layer 404, or disposed on the absorber layer 404 opposite the solid layer 402) that reflects photons back to the solid layer 402 and/or the absorber layer 404. The reflection layer may, for example, reflect un-upconverted infrared light back to the solid layer 402 in order to increase the proportion of infrared light that is upconverted by the photon upconverters.

The nanoparticles 400 may be incorporated into the solid layer 402 with a density of, for example, between about 10¹¹ and about 10¹² nanoparticles per liter of the solid layer 402, between about 10¹² and about 10¹³ nanoparticles per liter of the solid layer 402, between about 10¹³ and about 10¹⁴ nanoparticles per liter of the solid layer 402, between about 10¹⁴ and about 10¹⁵ nanoparticles per liter of the solid layer 402, between about 10¹⁵ and about 10¹⁶ nanoparticles per liter of the solid layer 402, between about 10¹⁶ and about 10¹⁷ nanoparticles per liter of the solid layer 402, between about 10¹¹ and about 10¹⁷ nanoparticles per liter of the solid layer 402, between about 10¹² and about 10¹⁶ nanoparticles per liter of the solid layer 402, between about 10¹³ and about 10¹⁵ nanoparticles per liter of the solid layer 402, or another density.

In some implementations, the chalcogenide nanoparticles 400 are configured to upconvert light generated by the light-sensitive material or by another portion of the device 401. For example, as the light-sensitive material of the absorber layer 404 absorbs light, the light-sensitive material may grow hot. In photovoltaic devices, this may cause a decrease in performance.

However, the light-sensitive material generates thermal infrared light 407, which may be upconverted by the nanoparticles 400 into upconverted thermal light 409. The upconverted thermal light 409 is absorbed by the light-sensitive material, which, in a photovoltaic device, leads to increased energy production. Waste heat is converted into useful electricity in a process of thermal management. The chalcogenide nanoparticles may be configured (e.g., by a placement of the chalcogenide nanoparticles, or by configured sizes of the chalcogenide nanoparticles) to upconvert the thermal infrared light 407.

Although some implementations described herein, including the implementation of FIG. 4, include both a solid layer 402 and an absorber layer 404, in some implementations of this disclosure an absorber layer or other auxiliary layer is not included. That is, a solid layer having embedded upconverting nanoparticles is itself an embodiment of the technologies described in this disclosure, even without an absorber or other receiver of the upconverted light. Upconversion characteristics of embedded nanoparticles can be, in various implementations, the same as or different from upconversion characteristics of the same nanoparticles suspended in solution or otherwise disposed.

Moreover, although FIG. 4 depicts the solid layer 402 disposed directly on the absorber layer 404, in some implementations the two layers are not directly in contact. In some implementations, one or more other layers may be interposed between the solid layer 402 and the absorber layer 404. In some implementations, a gap exists between the two layers.

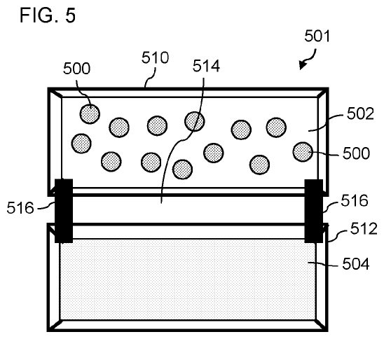

For example, FIG. 5 shows a device 501 including chalcogenide nanoparticles 500 (examples of a photon upconverter) embedded in a solid layer 502. The solid layer 502 is contained in a frame 510 that is attached to module 512 containing an absorber layer 504 including a light-sensitive material, with a gap 514 disposed between the two layers. Light may be upconverted by the nanoparticles 500 and absorbed in the absorber layer 504 as described above in reference to FIG. 4.

The frame 510 may be separably attachable and detachable to the module 512. For example, the frame 510 may include clips, bolts, or other attachment elements 516 configured to attach the frame 510 to the module 512.

In some implementations, the module 512 is a photovoltaic module, for example, a roof-based solar module. The frame 510 may be acquired separately from the module 512 (for example, as an aftermarket addon-on to the existing module 512). The frame 510 is attached to the module 512 between the module 512 and incoming sunlight, and the chalcogenide nanoparticles 500 in the frame 510 upconvert lower-energy light in the solar spectrum into higher-energy light that is more readily absorbed by the light-sensitive material in the absorber layer 504 of the module 512.

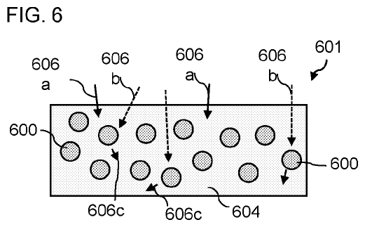

In some implementations, as shown in device 601 of FIG. 6, chalcogenide nanoparticles 600 are embedded directly in an absorber layer 604 that includes a light-sensitive material. For example, the chalcogenide nanoparticles 600 may be embedded in the light-sensitive material.

Some incoming light 606a (for example, incoming visible light) is absorbed directly by the light-sensitive material. Other incoming light 606b (for example, incoming infrared light) is absorbed more weakly by the light-sensitive material, or not absorbed by the light-sensitive material. However, the nanoparticles 600 upconvert the light 606b into output light 606c, which may be, for example, visible light. The output light 606c is absorbed in the absorber layer 604 by the light-sensitive material.

The implementation of FIG. 6 may be included in, for example, a solar cell or photosensor in which the light-sensitive material is an organic light absorber or a hybrid organic-inorganic perovskite light absorber. These materials may deposited onto substrates in processes including spin-coating, printing, and spray-coating. In some implementations, the nanoparticles 600 are mixed into the light-sensitive material before the light-sensitive material is deposited as part of device fabrication.

In some implementations, the chalcogenide nanoparticles are distributed in multiple materials or portions of a device. The chalcogenide nanoparticles may be distributed throughout a bulk heterojunction of an organic photovoltaic device, for example, throughout both of the two materials forming the bulk heterojunctions. In some implementations, the chalcogenide nanoparticles are distributed in a spacer layer of a solar cell.

In some implementations, chalcogenide nanoparticles are placed inside a patient for medical applications, including imaging (including microscopy) and phototherapeutic treatment.

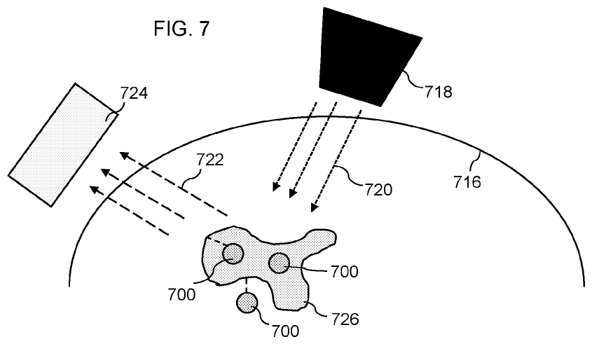

As shown in FIG. 7, chalcogenide nanoparticles 700 are inside a patient 716. A light source 718 illuminates the nanoparticles 700, which upconvert the input light 720 and generate higher-energy output light 722. The higher-energy output light 722 is absorbed by light-sensitive material of a detector 724, with the detector producing signals usable to produce an image of tissue inside the patient 716.

In some implementations, the light source 718 includes one or more low-threshold continuous-wave laser. In some implementations, the light source 718 includes one or more light-emitting diodes. In some implementations, the light source 718 emits lights of a single wavelength. In some implementations, the light source 718 emits light of multiple wavelengths. In some implementations, the light source 718 emits light that the nanoparticles 700 are configured to upconvert, e.g., by a selected diameter of the nanoparticles 700.

In existing technology, an intensity of light required to image inside a patient is sometimes prohibitive. Because light is absorbed by the body of the patient, it may be difficult to receive a sufficiently strong detected signal. However, increasing the intensity of input light (in order to correspondingly increase an intensity of detected light) risks injuring the patient or altering the tissue that is to be imaged. In addition, higher-energy light (for example, visible light) may be absorbed more strongly by tissue than lower-energy light (for example, infrared light).

The chalcogenide nanoparticles 700 may at least partially remedy this problem. The input light 720 may be higher-wavelength, lower-energy light (for example, infrared light), which may be absorbed less strongly by tissue than other light, e.g., the output light 722. Therefore, the input light 720 can penetrate more deeply into the patient 716 without being fully absorbed.

Inside the patient, the input light 720 is upconverted by the nanoparticles 700 into lower-wavelength, higher-energy output light 722 (for example, visible light). Because the output light 722 only needs to travel half the distance of the total light path (ie, only out of the patient, not both in and out of the patient), absorption of the output light 722 is reduced, and it may be collected by the detector 724 positioned outside the patient.

In some implementations, the detector 724 is a visible light detector having higher sensitivity than comparable infrared light detectors. In such implementations, upconversion of the light from infrared to visible not only may decrease absorption but also may increase effective detector sensitivity to the light.

Because higher-energy light has a smaller wavelength than lower-energy light, the diffraction limit for the output light 722 may be smaller than for the input light 720. Therefore, upconversion may increase the resolution of microscopy images derived from the detection of the light, compared to if light having the same wavelength as the input light 720 had been collected.

In the example of FIG. 7, the chalcogenide nanoparticles 700 are either bound to a cell 726, or endocytosed inside the cell 726, or both. The small size (for example, 20 nm diameter) of the chalcogenide nanoparticles 700 allows them to enter the cell 726 without damage to the cell 726.

In some implementations, the chalcogenide nanoparticles 700 are bound to a compound for applications in a patient. For example, the nanoparticles may be coated with a coating that allows the ingestion or in-vivo placement of the nanoparticles. In some implementations, the nanoparticles are bound to a compound that targets a particular other compound for binding. For example, the nanoparticles may be bound to a compound that binds to a particular type of tissue, cell, or pathogen.

In some implementations, the nanoparticles are bound to a drug.

In some implementations, the nanoparticles are placed inside a patient for purposes of phototherapy. The nanoparticles are positioned near a target site for irradiation. Input light is directed at the nanoparticles, which upconvert the light, and a portion of the output light strikes the target site. The higher-energy (output) light may therefore be directed mostly at or near the target site, rather than being absorbed by tissue all the way from outside the patient to the target site.

As described above, the chalcogenide nanoparticles are entirely or substantially non-toxic, allowing for their in-vivo use. Other upconverting materials may be more toxic and therefore unusable, or less practically usable, in the medical applications described herein.

In some implementations, chalcogenide nanoparticles are suspended in a solution. The solution may be configured (e.g., have a particular polarity, or have a particular chemical interaction with the chalcogenide nanoparticles) in order to improve the upconversion properties of the chalcogenide nanoparticles.

“Infrared light,” as used in this disclosure (e.g., in regard to optical excitation and/or possible transparencies of layers such as solid layers in which nanoparticles are embedded) includes at least light having wavelengths from 700 nm to 1 mm. In some implementations, properties relating to infrared light may specifically hold for near-infrared light, e.g., light having a wavelength between 800 nm and 2000 nm. For example, layers, such as solid layers in which nanoparticles are embedded, may be transparent to near-infrared light, and nanoparticles may upconvert near-infrared light into visible light.

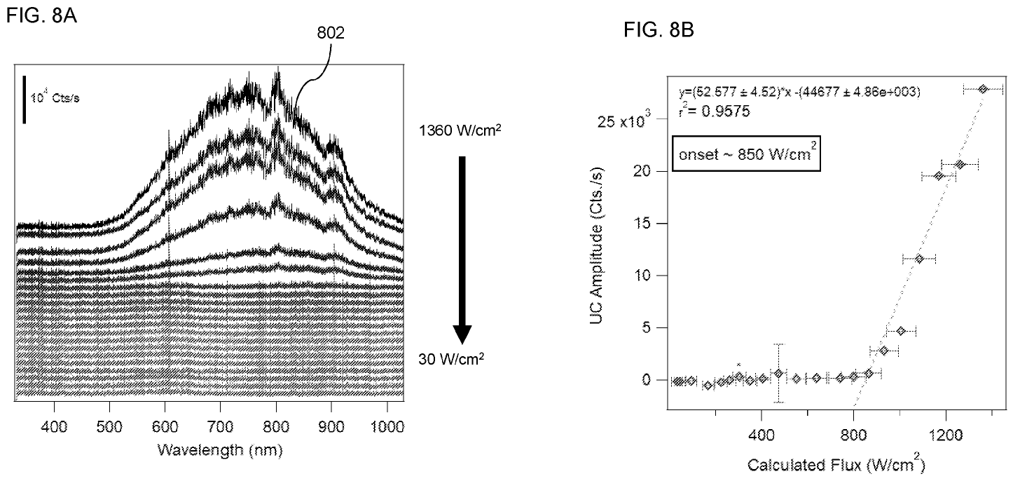

FIGS. 8A-8B show experimental data of upconverting Cu₂₋ₓSe nanoparticles embedded in a polymeric film, excited by a 1064 nm CW laser. The upconversion spectra (e.g., upconversion spectrum 802) observed for these films are substantially similar to upconversion spectra observed for the Cu₂₋ₓSe nanoparticles when suspended in solution.

As shown in FIG. 8A, upconversion by the Cu₂₋ₓSe nanoparticles results in appreciable upconverted output light from about 500 nm to over 1000 nm for excitation powers of greater than about 1000 W/cm². The internal quantum yield for at least some excitation powers over the onset excitation power (here, at least for an excitation power of 1360 W/cm²) is about 2%. In various implementations, the internal quantum yield of chalcogenide nanoparticles (including nanoparticles embedded in a solid layer) may be at least 10%, at least 5%, at least 3%, at least 2%, at least 1.5%, at least 1%, or another value, for excitation powers over the onset excitation power. The internal quantum yield may, in some implementations, be less than 20%, less than 15%, less than 10%, less than 5%, or another value.

As shown in FIG. 8B, which aggregates upconversion amplitude based on the data of FIG. 8A, the onset excitation power for the embedded Cu₂₋ₓSe nanoparticles is about 850 W/cm². In various implementations, the onset excitation power for chalcogenide nanoparticles (including nanoparticles embedded in a solid layer) may be less than 2000 W/cm², less than 1500 W/cm², less than 1200 W/cm², less than 1000 W/cm², or another value. The onset excitation power may be at least 100 W/cm², at least 200 W/cm², at least 400 W/cm², or another value.

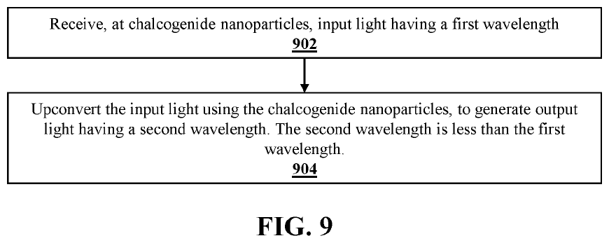

FIG. 9 shows an example method 900 according to some implementations of this disclosure. Input light having a first wavelength is received at chalcogenide nanoparticles (902). The input light is upconverted using the chalcogenide nanoparticles (904), to generate output light having a second wavelength. The second wavelength is less than the first wavelength.

Therefore, in accordance with the various embodiments of the disclosure, chalcogenide nanoparticles are used to upconvert light with advantages over other upconverting materials.

Other implementations not specifically described herein are also within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

It should be noted that any of the above-noted inventions may be provided in combination or individually. Elements of different embodiments described herein may be combined to form other embodiments not specifically set forth above.

While operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

1. A device comprising:
chalcogenide nanoparticles; and
a light-sensitive material configured to absorb upconverted light generated by the chalcogenide nanoparticles.

2. The device of claim 1, wherein the chalcogenide nanoparticles are copper selenide nanoparticles.

3. The device of claim 2, wherein the copper selenide nanoparticles are hole-doped Cu₂₋ₓSe nanoparticles.

4. The device of claim 1, wherein the chalcogenide nanoparticles are embedded in a solid layer transparent to at least one of infrared light and visible light.

5.-8. (canceled)

9. The device of claim 1, wherein the light-sensitive material is a light absorber in a solar cell or a photosensor.

10. The device of claim 1, wherein the chalcogenide nanoparticles each have a diameter between about 10 nanometers and about 30 nanometers.

11. The device of claim 1, wherein the chalcogenide nanoparticles upconvert infrared light into visible light.

12. The device of claim 1, wherein the chalcogenide nanoparticles comprise ligands or small molecules bound to the chalcogenide nanoparticles.

13. The device of claim 1, wherein the chalcogenide nanoparticles are embedded in the light-sensitive material.

14. The device of claim 13, wherein the light-sensitive material is a perovskite light absorber or an organic light absorber.

15. (canceled)

16. The device of claim 1, wherein the device is part of a solar cell or a photosensor.

17. (canceled)

18. The device of claim 1, wherein the chalcogenide nanoparticles are bound to a coating suitable for placement into a patient undergoing medical treatment.

19. The device of claim 18, wherein the coating is configured to bind to a cell inside the patient.

20. The device of claim 18, wherein the coating is bound to a drug.

21. (canceled)

22. (canceled)

23. (canceled)

24. A method comprising:
receiving, at chalcogenide nanoparticles, input light having a first wavelength; and
upconverting the input light using the chalcogenide nanoparticles, to generate output light having a second wavelength, wherein the second wavelength is less than the first wavelength.

25.-34. (canceled)

35. A device comprising:
a transparent material, the transparent material being transparent to at least one of infrared light and visible light, and
chalcogenide nanoparticles embedded in the transparent material.

36. The device of claim 35, wherein the transparent material comprises a glass or a polymer.

37.-39. (canceled)

40. The device of claim 35, further comprising a frame containing the transparent material,
wherein the frame is configured to be attached to a solar module.

41. The device of claim 35, wherein an internal quantum yield of the chalcogenide nanoparticles embedded in the transparent material is at least about 2% for 1064 nm input light.

42. The device of claim 35, wherein an onset power density of the chalcogenide nanoparticles embedded in the transparent material is less than 1000 W/cm² for 1064 nm input light.
